(19) United States
(12) Patent Application Publication
(10) Pub. No.: US 2011/0112039 A1
(43) Pub. Date: May 12, 2011

(54) Polysaccharides comprising carboxyl functional groups substituted via esterification by a hydrophobic alcohol

(75) Inventors: Richard CHARVET, Rillieux la Pape (FR); Remi SOULA, Lyon (FR); Olivier SOULA, Meyzieu (FR)

(73) Assignee: ADOCIA, LYON (FR)

(21) Appl. No.: 12/943,425

(22) Filed: Nov. 10, 2010

Related U.S. Application Data
(60) Provisional application No. 61/272,849, filed on Nov. 10, 2009.

(30) Foreign Application Priority Data
Nov. 10, 2009 (FR) 09 05409

(51) Int. Cl. C08B 37/00; C08B 37/02; A61K 38/02; A61K 38/14

(52) U.S. Cl. 514/20.9; 536/123.1; 536/119; 536/118; 536/53; 536/120; 536/112; 536/123.12; 514/1.1

(57) ABSTRACT

Polysaccharide including carboxyl functional groups. The polysaccharide being chosen from the group of anionic synthetic polysaccharides including 1,6 bonds obtained from neutral polysaccharides of which at least one of a carboxyl functional groups is esterified by a hydrophobic alcohol (-Ah) (residue of a hydrophobic alcohol). The hydrophobic alcohol (Ah) being grafted or bonded to the anionic polysaccharide by a function F (ester function), which results from coupling between the carboxylate function of the anionic polysaccharide and hydroxyl function of the hydrophobic alcohol. Carboxyl functions of anionic polysaccharide, which are not substituted, are in the form of carboxylate of a cation. The polysaccharide including carboxyl functional groups are amphiphilic at neutral pH.

It also relates to its use for the preparation of pharmaceutical compositions and the pharmaceutical compositions comprising a polysaccharide and at least one active principle.

The present invention relates to novel biocompatible polymers based on polysaccharides comprising carboxyl functional groups which may be useful, in particular for administering active principle(s) (AP) to humans or to animals for a therapeutic and/or prophylactic purpose.

Anionic dextrans and pullulans comprising carboxyl functional groups have, due to their structure and their biocompatibility, a particular advantage in pharmacy and more particularly in the field of stabilizing protein active principles by the formation of complexes.

Hydrophobic alcohols have an advantage in the formulation of pharmaceutical active principles, especially due to their hydrophobic nature that makes it possible to adjust the hydrophobicity of the polymers onto which they may be grafted and due to their biocompatibility.

Their biocompatibility is excellent insofar as they play a role in numerous biochemical processes and are present in esterified form in most tissues.

It is known to those skilled in the art that it is difficult to graft an alcohol onto a polysaccharide comprising carboxyl functional groups since it is difficult to be selective between the hydroxyl functions of the polysaccharide and the hydroxyl function of the hydrophobic alcohol. At the moment of grafting, the alcohols of the polymer may enter into competition with the alcohol of the graft if it is not desired to make use of techniques for protection/deprotection of the alcohols of the polymer and this secondary reaction results in the crosslinking of the polymer chains as is described in Zhang, R. et al., Biomaterials 2005, 26, 4677.

The difficulty in grafting hydrophobic alcohols to dextrans bearing carboxylate functions has, in particular, been sidestepped by grafting hydrophobic acids directly to the hydroxyl functions of the dextran. This has been carried out with activated derivatives of fatty acids, such as anhydrides (Novak L J, Tyree J T (1960) U.S. Pat. No. 2,954,372), acid chlorides, N-acyl ureas (Nichifor, Marieta et al., Eur. Polym. J. 1999, 35, 2125-2129), etc. These methods have only been used with neutral polysaccharides since these methods are not compatible with the presence of carboxylate functions on the polysaccharide.

Other, researchers have used non-anionic polysaccharides in order to be able to graft hydrophobic alcohols. Akiyoshi et al., for example, converted nucleophilic cholesterol to an electrophilic derivative (Biomacromolecules 2007, 8, 2366-2373). This electrophilic derivative of cholesterol was able to be grafted to the alcohol functions of pullulan or of mannan, neutral polysaccharides. This strategy also cannot be used with polysaccharides comprising carboxyl functional groups.

A recent review of dextran-based functional polymers (Heinze, T. et al., Adv. Polym. Sci. 2006, 205, 199-291) mentions modifications by hydrophobic acids, inter alfa, but does not mention dextran functionalized by hydrophobic alcohols.

Patent applications FR 08 505506, published under the number FR 2 936 800, and WO 2009/127940 describe carboxylated polysaccharides grafted by hydrophobic alcohols by means of a linker comprising an amine function capable of forming an amide bond with a carboxyl function of the polysaccharide. This solution, although it makes it possible to attain compounds of interest, comprises the drawback of introducing an additional amide function into the polysaccharide which may influence the formation and the stability of polysaccharide/active principle complexes.

The present invention relates to novel amphiphilic polysaccharide derivatives comprising carboxyl functional groups partly substituted by at least one hydrophobic alcohol. These novel polysaccharide derivatives comprising carboxyl functional groups have good biocompatibility and their hydrophobicity can easily be adjusted without impairing the biocompatibility or their stability.

It also relates to a method of synthesis that makes it possible to solve the synthesis problems mentioned above by using tosylated derivatives of hydrophobic alcohol. This method made it possible to obtain polysaccharides comprising carboxyl functional groups partly substituted by hydrophobic alcohols.

The invention therefore relates to polysaccharides comprising carboxyl functional groups, said polysaccharide being chosen from the group of anionic synthetic polysaccharides comprising, 1,6 bonds obtained from neutral polysaccharides, on which at least 15 carboxyl functional groups per 100 saccharide units have been grafted, of which at least one of said groups is substituted by a hydrophobic alcohol derivative, denoted by Ah:
said hydrophobic alcohol (Ah) being grafted or bonded to the anionic polysaccharide by a function F, said function F resulting from the coupling between the carboxylate function of the anionic polysaccharide and hydroxyl function of the hydrophobic alcohol, the carboxyl functions of the anionic polysaccharide that are not substituted being in the form of carboxylate of a cation, preferably an alkali metal cation such as Na⁺ or K⁺;
   F being an ester function;
Ah being a residue of a hydrophobic alcohol;
said polysaccharide comprising carboxyl functional groups being amphiphilic at neutral pH.

According to the invention, the polysaccharide comprising carboxyl functional groups partly substituted by hydrophobic alcohols is chosen from polysaccharides comprising carboxyl functional groups of general formula I:

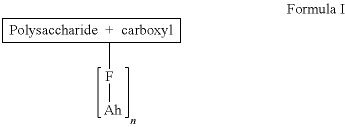

in which n represents the molar fraction of the carboxyl functions of the polysaccharide that are substituted by F-Ah and is between 0.01 and 0.7;

F and Ah corresponding to the definitions given above, and when the carboxyl function of the polysaccharide is not substituted by F-Ah, then the carboxyl functional group or groups of the polysaccharide are carboxylates of a cation, preferably an alkali metal cation such as Na⁺ or K⁺.

In one embodiment, the polysaccharides comprising carboxyl functional groups are synthetic polysaccharides obtained from neutral polysaccharides, onto which at least 15 carboxyl functional groups per 100 saccharide units have been grafted, of general formula II

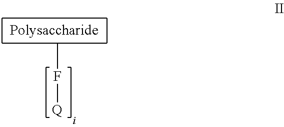

the natural polysaccharides being chosen from the group of polysaccharides, the bonds of which between the glycoside monomers comprise (1,6) bonds;

L being a bond that results from the coupling between the linker Q and an —OH function of the polysaccharide and being either an ester, thionoester, carbonate, carbamate or ether function;

i represents the molar fraction of the L-Q substituents per saccharide unit of the polysaccharide;

Q being chosen from the radicals of general formula III:

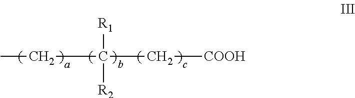

in which:

1≦a+b+c≦6, and

0≦a≦3,

0≦b≦3,

0≦c≦3,

R₁ and R₂, which are identical or different, are chosen from the group constituted by —H, linear or branched C1 to C3 alkyl, —COOH and the radical

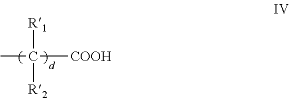

of formula IV in which:

1≦d≦3, and

R′₁ and R′₂, which are identical or different, are chosen from the group constituted by —H and a linear or branched C1 to C3 alkyl group.

In one embodiment, a+b+c≦5.

In one embodiment, a+b+c≦4.

In one embodiment, n is between 0.02 and 0.5.

In one embodiment, n is between 0.05 and 0.3.

In one embodiment, n is between 0.1 and 0.2.

In one embodiment, the polysaccharide is chosen from the group constituted by polysaccharides, the bonds of which between the glycoside monomers comprise (1,6) bonds.

In one embodiment, the polysaccharide is chosen from the group constituted by dextran and pullulan.

In one embodiment, the polysaccharide chosen from the group constituted by polysaccharides, the bonds of which between the glycoside monomers comprise (1,6) bonds, is dextran.

In one embodiment, the polysaccharide is chosen from the group constituted by polysaccharides, the bonds of which between the glycoside monomers comprise (1,6) bonds and (1,4) bonds.

In one embodiment, the polysaccharide chosen from the group constituted by polysaccharides, the bonds of which between the glycoside monomers comprise (1,6) bonds and (1,4) bonds, is pullulan.

In one embodiment, the polysaccharide according to the invention is characterized in that the L-Q radical is chosen from the group constituted by the following radicals, L having the meaning given above:

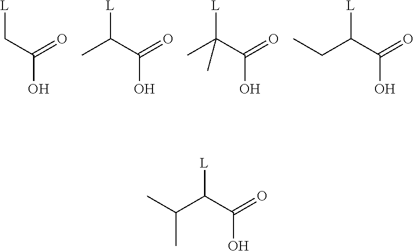

In one embodiment, the polysaccharide according to the invention is characterized in that the L-Q radical is chosen from the group constituted by the following radicals, L having the meaning given above:

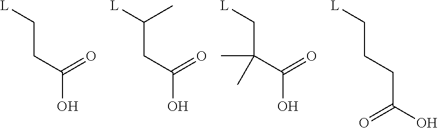

In one embodiment, the polysaccharide according to the invention is characterized in that the L-Q radical is chosen from the group constituted by the following radicals, L having the meaning given above:

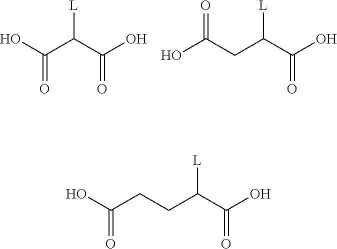

In one embodiment, i is between 0.15 and 2.

In one embodiment, i is between 0.3 and 1.5.

In one embodiment, the hydrophobic alcohol is chosen from fatty alcohols.

In one embodiment, the hydrophobic alcohol is chosen from alcohols constituted of an unsaturated or saturated, branched or unbranched, alkyl chain comprising from 4 to 18 carbons.

In one embodiment, the hydrophobic alcohol is chosen from alcohols constituted of an unsaturated or saturated, branched or unbranched, alkyl chain comprising from 6 to 18 carbons.

In one embodiment, the hydrophobic alcohol is chosen from alcohols constituted of an unsaturated or saturated, branched or unbranched, alkyl chain comprising from 8 to 16 carbons.

In one embodiment, the hydrophobic alcohol is octanol.

In one embodiment, the hydrophobic alcohol is 2-ethylbutanol.

In one embodiment, the fatty alcohol is chosen from myristyl, cetyl, stearyl, cetearyl, butyl, oleyl and lanolin alcohols.

In one embodiment, the hydrophobic alcohol is chosen from cholesterol derivatives.

In one embodiment, the cholesterol derivative is cholesterol.

In one embodiment, the hydrophobic alcohol is chosen from menthol derivatives.

In one embodiment, the hydrophobic alcohol is menthol in its racemic form.

In one embodiment, the hydrophobic alcohol is the D isomer of menthol.

In one embodiment, the hydrophobic alcohol is the L isomer of menthol.

In one embodiment, the hydrophobic alcohol is chosen from tocopherols.

In one embodiment, the tocopherol is alpha-tocopherol.

In one embodiment, the alpha-tocopherol is the racemate of alpha-tocopherol.

In one embodiment, the tocopherol is the D isomer of alpha-tocopherol.

In one embodiment, the tocopherol is the L isomer of alpha-tocopherol.

In one embodiment, the hydrophobic alcohol is chosen from alcohols bearing an aryl group.

In one embodiment, the alcohol bearing an aryl group is chosen from benzyl alcohol and phenethyl alcohol.

The polysaccharide may have a degree of polymerization m between 10 and 10 000.

In one embodiment, it has a degree of polymerization m between 10 and 1000.

In another embodiment, it has a degree of polymerization m between 10 and 500.

The invention also relates to the synthesis of polysaccharides comprising carboxyl functional groups that are partly substituted according to the invention.

Said synthesis comprises a step of obtaining an intermediate Ah-OTs and a step of grafting this tosylated intermediate to a carboxyl function of a polysaccharide, Ah corresponding to the definitions given above.

In one embodiment, a step for functionalizing the polysaccharide with at least 15 carboxyl functional groups per 100 saccharide units is carried out by grafting compounds of formula Q-L′, L′ being an anhydride, halide, tosylate, carboxylic acid, thio acid or isocyanate function, to at least 15 alcohol functions per 100 saccharide units of the polysaccharide, Q and L corresponding to the definitions given above.

In one embodiment, the tosylated intermediate of formula Ah-OTs is obtained by reaction of the hydrophobic alcohol Ah with a tosyl derivative according to the procedure described by Morita et al. (Morita, J.-I. et al., Green Chem. 2005, 7, 711).

Preferably, the step of grafting the tosylated intermediate to an acid function of the polysaccharide is carried out in an organic medium.

The invention also relates to the use of the functionalized polysaccharides according to the invention for the preparation of pharmaceutical compositions as described previously.

The invention also relates to a pharmaceutical composition comprising one of the polysaccharides according to the invention as described previously and at least one active principle.

The invention also relates to a pharmaceutical composition according to the invention as described previously, characterized in that the active principle is chosen from the group constituted by proteins, glycoproteins, peptides and non-peptide therapeutic molecules.

The expression “active principle” is understood to mean a product in the form of a single chemical entity or in the form of a combination having a physiological activity. Said active principle may be exogenous, that is to say that it is introduced by the composition according to the invention. It may also be endogenous, for example growth factors which will be secreted in a wound during the first phase of healing and which will be able to be retained on said wound by the composition according to the invention.

Depending on the pathologies targeted, the composition is intended for a local or systemic treatment.

In the case of local and systemic releases, the methods of administration envisioned are intravenous, subcutaneous, intradermal, transdermal, intramuscular, oral, nasal, vaginal, ocular, buccal, pulmonary, etc. administrations.

The pharmaceutical compositions according to the invention are either in liquid form, in aqueous solution, or in the form of a powder, an implant or a film. They also comprise the conventional pharmaceutical excipients well known to those skilled in the art.

Depending on the pathologies and on the methods of administration, the pharmaceutical compositions will advantageously be able to comprise, in addition, excipients that make it possible to formulate them in the form of a gel, sponge, injectable solution, drinkable solution, Lyoc (lyophilized tablet), etc.

The invention also relates to a pharmaceutical composition according to the invention as described previously, characterized in that it can be administered in the form of a stent, a film or “coating” of implantable biomaterials, or an implant.

### EXAMPLE 1

### Sodium dextranmethylcarboxylate partially esterified by octanol

### Polymer 1

1-Octyl p-toluenesulfonate is obtained according to the process described in the publication (Morita, J.-I. et al., Green Chem, 2005, 7, 711).

32 g (i.e. 0.59 mol of hydroxyl functions) of dextran having a weight-average molecular weight of around 10 kg/mol (Bachem) are dissolved in water to a concentration of 230 g/l. Added to this solution are 60 ml of 10 N NaOH (0.59 mol NaOH). The mixture is brought to 35° C., then 92 g (0.79 mol) of sodium chloroacetate are added. The temperature of the reaction medium is brought to 60° C. at a rate of 0.5° C./min then maintained at 60° C. for 100 minutes. The reaction medium is diluted with 800 ml of water, neutralized with acetic acid and purified by ultrafiltration through a 5 kD PES membrane against 6 volumes of water. The final solution is assayed by solids content in order to determine the polymer concentration; then assayed by acid/base titration in 50/50 (V/V) water/acetone in order to determine the degree of methylcarboxylate substitution.

According to the solids content: [polymer]=47.8 mg/g.

According to the acid/base titration: the degree of substitution of the hydroxyl functions by methylcarboxylate functions is 1.09 per saccharide unit.

The solution of sodium dextranmethylcarboxylate is passed over a(n) (anionic) Purolite resin in order to obtain an aqueous solution of dextranmethyl-carboxylic acid, the pH of which is raised to 7.1 by adding an aqueous (40%) solution of tetrabutylammonium hydroxide (Sigma), and the solution is then lyophilized for 18 hours.

20 g of tetrabutylammonium dextranmethyl-carboxylate (45 mmol methylcarboxylate functions) are dissolved in DMF at a concentration of 120 g/l, then heated at 40° C. A solution of 2.37 g of 1-octyl p-toluenesulfonate (8.3 mmol) in 12 ml of DMF is then added to the polymer solution. The medium is then maintained at 40° C. for 5 hours. The solution is ultra-filtered through a 10 kD PES membrane against 15 volumes of 0.9% NaCl solution, then 5 volumes of water. The concentration of the polymer solution is determined by solids content. A fraction of solution is lyophilized and analyzed by ¹H NMR in D₂O in order to determine the rate of acid functions converted to an ester of 1-octanol.

According to the solids content: [Polymer 1]=20.2 mg/g

According to the ¹H NMR: the molar fraction of acids esterified by the 1-octanol per saccharide unit is 0.17.

### EXAMPLE 2

### Sodium dextranmethylcarboxylate partially esterified by dodecanol

### Polymer 2

1-Dodecyl p-toluenesulfonate is obtained according to the process described in the publication (Morita, J.-I. et al., Green Chem, 2005, 7, 711).

Via a process similar to that described in example 1, using a dextran having a weight-average molecular weight of around 10 kg/mol (Pharmacosmos) a sodium dextranmethylcarboxylate partially esterified by dodecanol is obtained.

According to the solids content: [Polymer 2]=18.7 mg/g.

According to the ¹H NMR: the molar fraction of acids esterified by the dodecanol per saccharide unit is 0.095.

### EXAMPLE 3

### Sodium dextranmethylcarboxylate partially esterified by 3,7-dimethyl-1-octanol

### Polymer 3

3,7-Dimethyl-1-octyl p-toluenesulfonate is obtained according to the process described in the publication (Morita, J.-I. et al., Green Chem, 2005, 7, 711).

Via a process similar to that described in example 1, using a dextran having a weight-average molecular weight of around 10 kg/mol (Pharmacosmos) a sodium dextranmethylcarboxylate partially esterified by 3,7-dimethyl-1-octanol is obtained.

According to the solids content: [Polymer 3]=14 mg/g.

According to the ¹H NMR: the molar fraction of acids esterified by the 3,7-dimethyl-1-octanol per saccharide unit is 0.19.

### EXAMPLE 4

### Sodium dextranmethylcarboxylate partially esterified by 2-hexyl-1-decanol

### Polymer 4

2-Hexyl-1-decyl p-toluenesulfonate is obtained according to the process described in the publication (Morita, J.-I. et al., Green Chem, 2005, 7, 711).

Via a process similar to that described in example 1, using a dextran having a weight-average molecular weight of around 10 kg/mol (Pharmacosmos) a sodium dextranmethylcarboxylate partially esterified by 2-hexyl-1-decanol is obtained.

According to the solids content: [Polymer 4]=20.5 mg/g.

According to the ¹H NMR: the molar fraction of acids esterified by the 2-hexyl-1-decanol per saccharide unit is 0.05.

### EXAMPLE 5

### Sodium dextran(2-ethyl)methylcarboxylate partially esterified by octanol

### Polymer 5

1-octyl p-toluenesulfonate is obtained according to the process described in the publication (Morita, J.-I. et al., Green Chem, 2005, 7, 711).

15 g (i.e. 0.28 mol of hydroxyl functions) of dextran having a weight-average molecular weight of around 40 kg/mol (Bachem) are dissolved in 14 ml of water. Next, 55.8 g (0.33 mol) of 2-bromobutyric acid and 37 ml of 10 N NaOH are added and the mixture is heated at 55° C. 46.3 ml of 10 N NaOH are added over 1 h and the medium is heated at 55° C. for 50 min. The reaction medium is diluted with 24 ml of water, neutralized with acetic acid and purified by ultrafiltration through a 5 kD PES membrane against 15 volumes of water. The final solution is assayed by solids content in order to determine the polymer concentration; then assayed by acid/base titration in 50/50 (V/V) water/acetone in order to determine the degree of (2-ethyl)methylcarboxylate substitution.

According to the solids content: [polymer]=46.6 mg/g.

According to the acid/base titration: the degree of substitution of the hydroxyl functions by (2-ethyl)methylcarboxylate functions is 0.43 per saccharide unit.

The solution of sodium dextran(2-ethyl)methylcarboxylate is passed over a(n) (anionic) Purolite resin in order to obtain an aqueous solution of dextran(2-ethyl)methylcarboxylic acid, the pH of which is raised to 7.1 by adding an aqueous (40%) solution of tetrabutylammonium hydroxide (Sigma), and the solution is then lyophilized for 18 hours.

13 g of tetrabutylammonium dextran(2-ethyl)-methylcarboxylate (18 mmol (2-ethyl)methylcarboxylate functions) are dissolved in DMF at a concentration of 100 g/l, then heated at 40° C. A solution of 0.6 g of 1-octyl p-toluenesulfonate (2.1 mmol) in 3 ml of DMF is then added to the polymer solution. The medium is then maintained at 40° C. for 5 hours. The solution is ultra-filtered through a 10 kD PES membrane against 15 volumes of 0.9% NaCl solution, then 5 volumes of water. The concentration of the polymer solution is determined by solids content. A fraction of solution is lyophilized and analyzed by ¹H NMR in D₂O in order to determine the rate of acid functions converted to an ester of 1-octanol.

According to the solids content: [Polymer 5]=24.2 mg/g

According to the ¹H NMR: the molar fraction of acids esterified by the 1-octanol per saccharide unit is 0.1.

### EXAMPLE 6

### Sodium dextransuccinate partially esterified by dodecanol

### Polymer 6

1-Dodecyl p-toluenesulfonate is obtained according to the process described in the publication (Morita, J.-I. et al., Green Chem, 2005, 7, 711).

Sodium dextransuccinate is obtained from dextran 40 according to the method described in the article by Sanchez-Chaves et al., (Sanchez-Chaves, Manuel et al., Polymer 1998, 39 (13), 2751-2757). The rate of acid functions per glycoside unit is 1.53 according to ¹H NMR in D₂O/NaOD.

Via a process similar to that described in example 1, a sodium dextransuccinate partially esterified by 1-dodecanol is obtained.

According to the solids content: [Polymer 6]=28.2 mg/g.

According to the ¹H NMR: the molar fraction of acids esterified by the 1-dodecanol per saccharide unit is 0.05.

### EXAMPLE 7

### Sodium dextran carbamate N-methylcarboxylate partially esterified by octanol

### Polymer 7

1-octyl p-toluenesulfonate is obtained according to the process described in the publication (Morita, J.-I. et al., Green Chem, 2005, 7, 711).

11.5 g (i.e. 0.21 mol of hydroxyl functions) of dextran having a weight-average molecular weight of around 10 kg/mol (Bachem) are dissolved in a DMF/DMSO mixture. The mixture is brought to 130° C. while stirring and 13.75 g (0.11 mol) of ethyl isocyanatoacetate are gradually introduced. The medium is then diluted with water and purified by diafiltration through a 5 kD PES membrane against 0.1N NaOH, 0.9% NaCl and water. The final solution is assayed by solids content in order to determine the polymer concentration; then assayed by acid/base titration in 50/50 (V/V) water/acetone in order to determine the degree of carboxylate charge substitution.

According to the solids content: [polymer]=38.9 mg/g.

According to the acid/base titration: the degree of substitution of the hydroxyl functions by carbamate N-methylcarboxylate functions is 1.08 per saccharide unit.

The solution of sodium dextran carbamate N-methylcarboxylate is passed over a(n) (anionic) Purolite resin in order to obtain an aqueous solution of dextran carbamate N-methylcarboxylic acid, the pH of which is raised to 7.1 by adding an aqueous (40%) solution of tetrabutylammonium hydroxide (Sigma), and the solution is then lyophilized for 18 hours.

12.1 g of tetrabutylammonium dextran carbamate N-methylcarboxylate (25 mmol tetrabutyl-ammonium carbamate N-methylcarboxylate functions) are dissolved in DMF at a concentration of 140 g/l, then heated at 40° C. A solution of 0.65 g of 1-octyl p-toluenesulfonate (3.2 mmol) in 27 ml of DMF is then added to the polymer solution. The medium is then maintained at 40° C. for 5 hours. The solution is ultra-filtered through a 10 kD PES membrane against 15 volumes of 0.9% NaCl solution, then 5 volumes of water. The concentration of the polymer solution is determined by solids content. A fraction of solution is lyophilized and analyzed by ¹H NMR in D₂O in order to determine the rate of acid functions converted to an ester of 1-octanol.

According to the solids content: [Polymer 7]=20.2 mg/g.

According to the ¹H NMR: the molar fraction of acids esterified by the 1-octanol per saccharide unit is 0.09.

### EXAMPLE 8

### Stabilization of a human polyclonal antibody with respect to mechanical stress

A test of stabilization of a human polyclonal antibody with respect to mechanical stress was developed in order to demonstrate the stabilizing power of the polysaccharides of the invention. An aqueous solution (375 μl) of polymer (1.33 mmol/l) is diluted with 75 μl of sodium chloride (1.5 M, Riedel-de-Haën). 375 μl of a solution of human polyclonal antibody (80 g/l, i.e. 0.53 mmol/l) is then added to the polymer solution in order to generate a final solution having an antibody concentration of 40 mg/ml for a polymer/antibody molar ratio of 2.

The polymer, polymer 1, according to the invention is used in this test. By way of comparison, a polymer described in patent application FR0805506 is also used in this test, the sodium dextranmethyl-carboxylate modified by 1-octanol glycinate, polymer 8.

The test consists in agitating, by upturning at 30 rpm, the formulations placed in 6 ml glass hemolysis tubes.

After upturning for 18 h, the visual appearance of the solution is noted and the optical density of the sample at 450 nm is measured.

The results are collated in the table below.

| | Visual aggregation | OD at 450 nm at |
|---|---|---|
| Solution | at 18 h | 18 h |
| Antibody alone | Limited | 0.38 |
| Polymer 1 | None | 0.26 |
| Polymer 8 | High | 1.58 |
| counter example | | |

This test makes it possible to demonstrate the improvement in the stability of a human polyclonal antibody in solution by the polymer according to the invention in a mechanical stress test. On the other hand, the sodium dextranmethylcarboxylate modified by 1-octanol glycinate stimulates this aggregation.

1. A polysaccharide comprising carboxyl functional groups, said polysaccharide being chosen from the group of anionic synthetic polysaccharides comprising 1,6 bonds obtained from neutral polysaccharides, on which at least 15 carboxyl functional groups per 100 saccharide units have been grafted, of which at least one of said carboxyl functional groups is esterified by a hydrophobic alcohol, denoted by Ah:
said hydrophobic alcohol (Ah) being grafted or bonded to the anionic polysaccharide by a function F, said function F resulting from the coupling between the carboxylate function of the anionic polysaccharide and hydroxyl function of the hydrophobic alcohol, the carboxyl functions of the anionic polysaccharide that are not substituted being in the form of carboxylate of a cation, preferably an alkali metal cation such as Na⁺ or K⁺;
F being an ester function;
Ah being a residue of a hydrophobic alcohol;
said polysaccharide comprising carboxyl functional groups being amphiphilic at neutral pH.

2. The polysaccharide as claimed in claim 1, wherein it is chosen from the polysaccharides of general formula I: 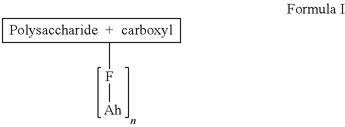
in which n represents the molar fraction of the carboxyl functions of the polysaccharide that are substituted by F-Ah and is between 0.01 and 0.7;
F and Ah corresponding to the definitions given above, and when the carboxyl function of the polysaccharide is not substituted by F-Ah, then the carboxyl functional group or groups of the polysaccharide are carboxylates of a cation, preferably an alkali metal cation such as Na⁺ or K⁺.

3. The polysaccharide as claimed in claim 1, wherein the synthetic polysaccharides obtained from neutral polysaccharides, on which at least 15 carboxyl functional groups per 100 saccharide units have been grafted, are chosen from the polysaccharides of general formula II. 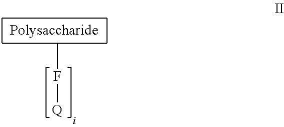
the natural polysaccharides being chosen from the group of polysaccharides partly constituted of glycoside monomers bonded by glycosidic bonds of (1,6) type;
L being a bond that results from the coupling between the linker Q and an —OH function of the polysaccharide and being either an ester, thionoester, carbonate, carbamate or ether function;
i represents the molar fraction of the L-Q substituents per saccharide unit of the polysaccharide;
Q being chosen from the radicals of general formula III: 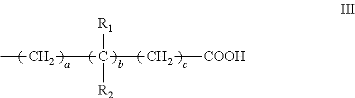
in which:
1≦a+b+c≦6,
0≦a≦3,
0≦b≦3,
0≦c≦3,
R₁ and R₂, which are identical or different, are chosen from the group constituted by —H, linear or branched C1 to C3 alkyl, —COOH and the radical 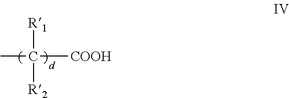
of formula IV in which:
1≦d≦3, and
R′₁ and R′₂, which are identical or different, are chosen from the group constituted by —H and a linear or branched C1 to C3 alkyl group.

4. The polysaccharide as claimed in claim 1, wherein the polysaccharide is chosen from the group constituted by dextran and pullulan.

5. The polysaccharide as claimed in claim 1, wherein the polysaccharide is dextran.

6. The polysaccharide as claimed in claim 1, wherein the polysaccharide is pullulan.

7. The polysaccharide as claimed in claim 1, wherein the L-Q radical is chosen from the group constituted by the following radicals: 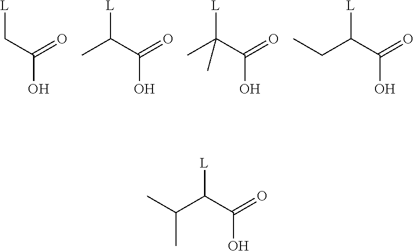
L being a bond that results from the coupling between the linker Q and an —OH function of the polysaccharide and being either an ester, thionoester, carbonate, carbamate or ether function.

8. The polysaccharide as claimed in claim 1, wherein the L-Q radical is chosen from the group constituted by the following radicals: 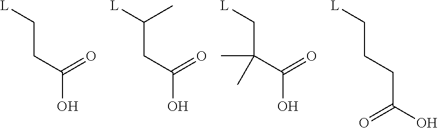
L being a bond that results from the coupling between the linker Q and an —OH function of the polysaccharide and being either an ester, thionoester, carbonate, carbamate or ether function.

9. The polysaccharide as claimed in claim 1, wherein the L-Q radical is chosen from the group constituted by the following radicals: 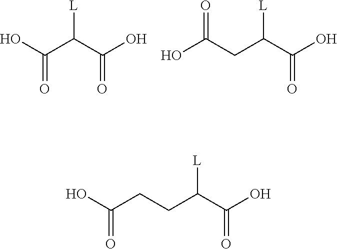
L being a bond that results from the coupling between the linker Q and an —OH function of the polysaccharide and being either an ester, thionoester, carbonate, carbamate or ether function.

10. The polysaccharide as claimed in claim 1, wherein the hydrophobic alcohol is chosen from fatty alcohols.

11. The polysaccharide as claimed in claim 1, wherein the hydrophobic alcohol is chosen from the alcohols constituted of an unsaturated or saturated alkyl chain comprising from 4 to 18 carbons.

12. The polysaccharide as claimed in claim 1, wherein the fatty alcohol is chosen from myristyl, cetyl, stearyl, cetearyl, butyl, oleyl and lanolin alcohols.

13. The polysaccharide as claimed in claim 1, wherein the hydrophobic alcohol is cholesterol.

14. The polysaccharide as claimed in claim 1, wherein the hydrophobic alcohol is menthol.

15. The polysaccharide as claimed in claim 1, wherein the hydrophobic alcohol is chosen from tocopherols, preferably alpha-tocopherol.

16. The polysaccharide as claimed in claim 1, wherein the hydrophobic alcohol is chosen from alcohols bearing an aryl group.

17. The polysaccharide as claimed in claim 16, wherein the alcohol bearing an aryl group is chosen from benzyl alcohol and phenethyl alcohol.

18. A method of preparing pharmaceutical compositions, the method comprising utilizing the functionalized polysaccharide of claim 1.

19. A pharmaceutical composition comprising a polysaccharide as claimed in claim 1 and at least one active principle.

20. The pharmaceutical composition as claimed in claim 19, wherein it can be administered by oral, nasal, vaginal or buccal administration.

21. The pharmaceutical composition as claimed in claim 19, wherein the active principle is chosen from the group constituted by proteins, glycoproteins, peptides and non-peptide therapeutic molecules.
